(19) United States
(12) Patent Application Publication
(10) Pub. No.: US 2023/0215519 A1
(43) Pub. Date: Jul. 6, 2023

(54) INDEXING OF CLINICAL BACKGROUND INFORMATION FOR ANATOMICAL RELEVANCY

(71) Applicant: MERATIVE US L.P., Ann Arbor, MI (US)

(72) Inventor: Tin Kam Ho, Millburn, NJ (US)

(21) Appl. No.: 17/569,467

(22) Filed: Jan. 5, 2022

(51) Int. Cl. G16H 10/20; G16H 10/60; G16H 15/00; G16H 30/20

(52) U.S. Cl. CPC G16H 10/20; G16H 10/60; G16H 15/00; G16H 30/20

(57) ABSTRACT

Methods and systems of determining relevancy of electronic health records to medical analysis objectives. One system includes an electronic processor configured to access electronic health records and extract medical summary data items from the records. The electronic processor is also configured to determine a set of semantic vectors, where each semantic vector represents a medical summary data item. The electronic processor is also configured to determine a set of anatomical semantic vectors. The electronic processor is also configured to determine a similarity score for each medical summary data item. The electronic processor is also configured to receive a medical study and determine a relevancy score for each medical summary data item, the relevancy score representing a relevancy of each medical summary data item to the medical study. The electronic processor is also configured to generate and transmit a notification to a reviewer of the medical study.

### FIELD

Embodiments described herein generally relate to indexing of clinical background information for anatomical relevancy.

### SUMMARY

Radiologists seeking to interpret a medical image associated with a patient may benefit from a compact summary of the patient’s clinically relevant information from, for example, electronic health records. Medical summary data items like chief complaints, past medical or surgical histories, and the like may provide hints, alerts, and explanations to what may be present in the current medical image. However, large accumulations of medical records may yield medical summary data items that may become relevant under different studies in a later time. Accordingly, there is a need to organize medical summary data items in a way that can support their use with maximum flexibility in any future study.

To solve these and other problems, embodiments described herein provide methods and systems for indexing of clinical background information for anatomical relevancy such that medical summary data items relevant to a current medical image may be automatically identified and provided to a reviewer of the current medical image. In particular, embodiments described herein uses an anatomical reference system to index the patient information summaries, which links the informative items, such as, for example, symptoms, diagnoses, current or past illnesses, and past surgeries, to critical body parts and major organs that are subject to common radiology studies. At the time of extraction, each medical summary data item is assigned a set of scores according to its relevance to the chosen dimensions of the reference frame. Each imaging view in popular radiological studies is also assigned a set of weights on the same reference dimensions. At retrieval time, medical summary data items relevant to each view may be re-ranked on demand using the weights and the scores together.

For example, one embodiment provides a system of determining relevancy of electronic health records to medical analysis objectives. The system includes an electronic processor configured to access a set of electronic health records associated with a patient. The electronic processor is also configured to extract a set of medical summary data items from the set of electronic health records. The electronic processor is also configured to determine a set of semantic vectors, each semantic vector representing a medical summary data item. The electronic processor is also configured to determine, using a set of anatomical concepts providing a reference frame, a set of anatomical semantic vectors, each anatomical semantic vector representing at least one anatomical concept. The electronic processor is also configured to determine, using the set of anatomical semantic vectors, a similarity score for each medical summary data item as a set of similarity scores, wherein the similarity score represents an association between each medical summary data item and an anatomical concept, wherein the set of similarity scores is determined using a function of a semantic vector representing the medical summary data item and the anatomical semantic vector representing the anatomical concept. The electronic processor is also configured to receive a medical study associated with the patient, the medical study associated with at least one anatomical concept. The electronic processor is also configured to determine a relevancy score for each medical summary data item as a set of relevancy scores, wherein the relevancy score represents a relevancy of each medical summary data item to the medical study. The electronic processor is also configured to generate and transmit a notification to a reviewer of the medical study, wherein the notification indicates at least one medical summary data item that is relevant to the medical study.

Another embodiment provides a method of determining relevancy of electronic health records to medical analysis objectives. The method includes generating, with an electronic processor, a reference frame including a plurality of anatomical reference vectors, each anatomical reference vector associated with an anatomical concept. The method also includes accessing, with the electronic processor, a set of electronic health records associated with a patient. The method also includes extracting, with the electronic processor, a set of medical summary data items from the set of electronic health records. The method also includes determining a set of semantic vectors, each semantic vector representing a medical summary data item. The method also includes determining, with the electronic processor, using the using the set of anatomical concepts providing the reference frame, a set of anatomical semantic vectors, each anatomical semantic vector representing at least one anatomical concept. The method also includes determining, with the electronic processor, using the set of anatomical semantic vectors, a similarity score for each medical summary data item as a set of similarity scores, wherein the similarity score represents an association between each medical summary data item and an anatomical concept, wherein the set of similarity scores is determined using a function of a semantic vector representing the medical summary data item and the anatomical semantic vector representing the anatomical concept. The method also includes receiving, with the electronic processor, a medical study associated with the patient, the medical study associated with at least one anatomical concept. The method also includes determining, with the electronic processor, a relevancy score for each medical summary data item as a set of relevancy scores, wherein the relevancy score represents a relevancy of each medical summary data item to the medical study. The method also includes generating and transmitting, with the electronic processor, a notification to a reviewer of the medical study, wherein the notification indicates at least one medical summary data item that is relevant to the medical study.

Another embodiment provides a non-transitory, computer-readable medium storing instructions that, when executed by an electronic processor, perform a set of functions. The set of functions includes accessing a set of electronic health records associated with a patient. The set of functions also includes extracting a set of medical summary data items from the set of electronic health records. The set of functions also includes determining a set of semantic vectors, each semantic vector representing a medical summary data item. The set of functions also includes determining, using a set of anatomical concepts providing a reference frame, a set of anatomical semantic vectors, each anatomical semantic vector representing at least one anatomical concept. The set of functions also includes determining, using the set of anatomical semantic vectors, a similarity score for each medical summary data item as a set of similarity scores, wherein the similarity score represents an association between each medical summary data item and an anatomical concept, wherein the set of similarity scores is determined using a fuction of a semantic vector representing the medical summary data item and the anatomical semantic vector representing the anatomical concept. The set of functions also includes receiving a radiology study associated with the patient, the medical study associated with at least one anatomical concept. The set of functions also includes determining a relevancy score for each medical summary data item as a set of relevancy scores, wherein the relevancy score represents a relevancy of each medical summary data item to the radiology study. The set of functions also includes generating and transmitting a notification to a reviewer of the medical study, wherein the notification indicates at least one medical summary data item that is relevant to the radiology study.

Other aspects of the embodiments described herein will become apparent by consideration of the detailed description and accompanying drawings.

### BRIEF DESCRIPTION OF THE DRAWINGS

FIG. 1 illustrates a system for determining relevancy of electronic health records to medical analysis objectives according to some embodiments.

FIG. 2 illustrates a server included in the system of FIG. 1 according to some embodiments.

FIG. 3 illustrates a method for determining relevancy of electronic health records to medical analysis objectives using the system of FIG. 1 according to some embodiments.

FIG. 4 illustrates an example anatomical reference system or frame according to some embodiments.

Other aspects of the embodiments described herein will become apparent by consideration of the detailed description.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used herein, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

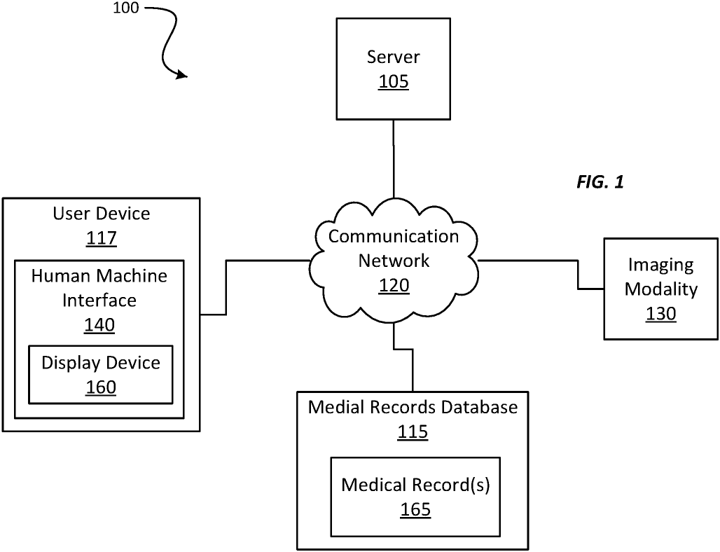

FIG. 1 schematically illustrates a system 100 for determining relevant medical data associated with a patient according to some embodiments. The system 100 includes a server 105, a medical records database 115, a user device 117, and an image modality 130. In some embodiments, the system 100 includes fewer, additional, or different components than illustrated in FIG. 1. For example, the system 100 may include multiple servers 105, medical records databases 115, user devices 117, image modalities 130, or a combination thereof.

The server 105, the medical records database 115, the user device 117, and the image modality 130 communicate over one or more wired or wireless communication networks 120. Portions of the communication network 120 may be implemented using a wide area network, such as the Internet, a local area network, such as a Bluetooth™ network or Wi-Fi, and combinations or derivatives thereof. Alternatively or in addition, in some embodiments, components of the system 100 communicate directly as compared to through the communication network 120. Also, in some embodiments, the components of the system 100 communicate through one or more intermediary devices not illustrated in FIG. 1.

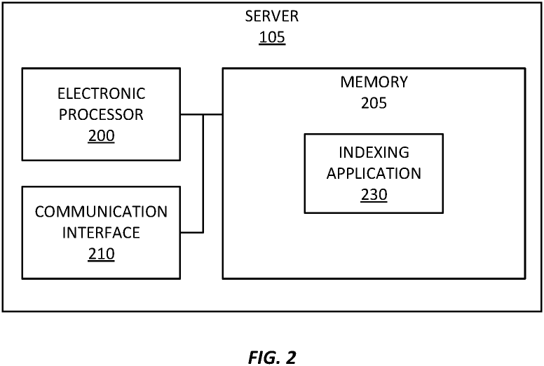

The server 105 is a computing device, which may serve as a gateway for the medical records database 115. For example, in some embodiments, the server 105 may be a PACS server. Alternatively, in some embodiments, the server 105 may be a server that communicates with a PACS server to access the medical records database 115. As illustrated in FIG. 2, the server 105 includes an electronic processor 200, a memory 205, and a communication interface 210. The electronic processor 200, the memory 205, and the communication interface 210 communicate wirelessly, over one or more communication lines or buses, or a combination thereof. The server 105 may include additional components than those illustrated in FIG. 2 in various configurations. The server 105 may also perform additional functionality other than the functionality described herein. Also, the functionality (or a portion thereof) described herein as being performed by the server 105 may be distributed among multiple devices, such as multiple servers included in a cloud service environment. In addition, in some embodiments, the user device 117 may be configured to perform all or a portion of the functionality described herein as being performed by the server 105.

The electronic processor 200 includes a microprocessor, an application-specific integrated circuit (ASIC), or another suitable electronic device for processing data. The memory 205 includes a non-transitory computer-readable medium, such as read-only memory (“ROM”), random access memory (“RAM”) (for example, dynamic RAM (“DRAM”), synchronous DRAM (“SDRAM”), and the like), electrically erasable programmable read-only memory (“EEPROM”), flash memory, a hard disk, a secure digital (“SD”) card, another suitable memory device, or a combination thereof. The electronic processor 200 is configured to access and execute computer-readable instructions (“software”) stored in the memory 205. The software may include firmware, one or more applications, program data, filters, rules, one or more program modules, and other executable instructions. For example, the software may include instructions and associated data for performing a set of functions, including the methods described herein.

For example, as illustrated in FIG. 2, the memory 205 may store an indexing application 230. In some embodiments, the indexing application 230 is a software application executable by the electronic processor 200. As described in more detail below, the electronic processor 200 executes the indexing application 230 to index or organize clinical background information or data (for example, a medical history associated with a patient) for anatomical relevancy such that medical summary data items relevant to a current medical image or study may be automatically identified and provided to a reviewer of the current medical image or study.

The communication interface 210 allows the server 105 to communicate with devices external to the server 105. For example, as illustrated in FIG. 1, the server 105 may communicate with the medical records database 115, the user device 117, the image modality 130, or a combination thereof through the communication interface 210. In particular, the communication interface 210 may include a port for receiving a wired connection to an external device (for example, a universal serial bus (“USB”) cable and the like), a transceiver for establishing a wireless connection to an external device (for example, over one or more communication networks 120, such as the Internet, local area network (“LAN”), a wide area network (“WAN”), and the like), or a combination thereof.

The user device 117 is also a computing device and may include a desktop computer, a terminal, a workstation, a laptop computer, a tablet computer, a smart watch or other wearable, a smart television or whiteboard, or the like. Although not illustrated, the user device 117 may include similar components as the server 105 (an electronic processor, a memory, and a communication interface). The user device 117 may also include a human-machine interface 140 for interacting with a user. The human-machine interface 140 may include one or more input devices, one or more output devices, or a combination thereof. Accordingly, in some embodiments, the human-machine interface 140 allows a user to interact with (for example, provide input to and receive output from) the user device 117. For example, the human-machine interface 140 may include a keyboard, a cursor-control device (for example, a mouse), a touch screen, a scroll ball, a mechanical button, a display device (for example, a liquid crystal display (“LCD”)), a printer, a speaker, a microphone, or a combination thereof. As illustrated in FIG. 1, in some embodiments, the human-machine interface 140 includes a display device 160. The display device 160 may be included in the same housing as the user device 117 or may communicate with the user device 117 over one or more wired or wireless connections. For example, in some embodiments, the display device 160 is a touchscreen included in a laptop computer or a tablet computer. In other embodiments, the display device 160 is a monitor, a television, or a projector coupled to a terminal, desktop computer, or the like via one or more cables.

Additionally, in some embodiments, to communicate with the server 110, the user device 117 may store a browser application or a dedicated software application executable by an electronic processor of the user device 117. The system 100 is described herein as providing a relevancy based indexing or organization service through the server 110. However, in other embodiments, the functionality (or a portion thereof) described herein as being performed by the server 110 may be locally performed by the user device 117. For example, in some embodiments, the user device 117 may store the indexing application 230.

The medical records database 115 stores a plurality of medical records 165 (for example, electronic heath records). In some embodiments, the medical records database 115 is combined with the server 105. Alternatively or in addition, the medical records 165 may be stored within a plurality of databases, such as within a cloud service. Although not illustrated in FIG. 1, the medical records database 115 may include components similar to the server 105, such as an electronic processor, a memory, a communication interface, and the like. For example, the medical records database 115 may include a communication interface configured to communicate (for example, receive data and transmit data) over the communication network 120.

The medical records 165 stored in the medical records database 115 includes medical or health related information associated with a patient. For example, the medical records 165 may be electronic health records associated with the patient, such as, for example, previous electronic health records associated with a medical history of the patient. Accordingly, the medical records 165 may provide clinical background information associated with a patient. The medical records 165 may include, for example, patient information summaries, medical case studies, imaging studies, medical reports, and the like. The medical records 165 (or electronic health records) may include one or more medical summary data items (for example, patient information summaries). A medical summary data item may include text, such as a phrase, a sentence, or a word. In some embodiments, the medical summary data item includes structured text, unstructured text, or a combination thereof. The medical summary data items may include, for example, a symptom, a diagnosis, a test result, a current illness, a past illness, information regarding a past procedure or surgery, family medical history information, and the like. In some embodiments, a memory of the medical records database 115 stores the medical records 165 and associated data (for example, metadata). For example, the medical records database 115 may include a picture archiving and communication system (“PACS”), a radiology information system (“RIS”), an electronic medical record (“EMR”) system, a hospital information system (“HIS”), an image study ordering system, and the like.

The imaging modality 130 provides imagery (for example, the medical images). The imaging modality 130 may include a computed tomography (CT), a magnetic resonance imaging (MRI), an ultrasound (US), another type of imaging modality, or a combination thereof. While the embodiments described herein are generally described in the context of radiology medical images, it should be understood that other images, such as pathology images, including gross specimen photos, microscopy slide images, and whole scanned slide datasets, may also be used. Other images, such as dermatology, intra-operative or surgery, or wound care photos or movies, may also be used. In some embodiments, the medical images or studies are transmitted from the imaging modality 130 to a PACS Gateway (for example, the server 105). Alternatively or in addition, in some embodiments, the medical images or studies are transmitted from the imaging modality 130 to the medical records database 115 (for example, as a new medical record).

A user may use the user device 117 to access, view, and interact with the medical records 165 (including one or more medical images or studies provided by the imaging modality 130). For example, the user may access the medical records 165 (for example, a medical study or image) from the medical records database 115 (through a browser application or a dedicated application stored on the user device 117 that communicates with the server 105) and view the medical records 165 (or medical study or image) on the display device 160 associated with the user device 117. A user may interact with the medical records 165 by accessing a new medical record 165 (for example, a medical image recently captured by the imaging modality 130) to read and review the new medical record 165 (as a new medical study), such as, for example, for diagnosing purposes, annotating purposes, and the like.

Radiologists seeking to interpret a medical image associated with a patient (such as a new medical study or record) may benefit from a compact summary of the patient’s clinically relevant information from, for example, electronic health records (for example, the medical records 165). Medical summary data items like chief complaints, past medical or surgical histories, and the like may provide hints, alerts, and explanations to what may be present in the current medical image (for example, the new medical study). However, large accumulations of medical records (for example, the medical records 165) may yield medical summary data items that may become relevant under different studies in a later time. Accordingly, there is a needed to organize medical summary data items in a way that can support their use with maximum flexibility in future studies. To solve these and other problems, the system 100 is configured to index clinical background information for anatomical relevancy such that medical summary data items relevant to a current medical image may be automatically identified and provided to a reviewer of the current medical image. In particular, in some embodiments, the methods and systems described herein use an anatomical reference system to index the patient information summaries, which links the informative items, such as, for example, symptoms, diagnoses, current or past illnesses, and past surgeries, to critical body parts and major organs that are subject to common radiology studies.

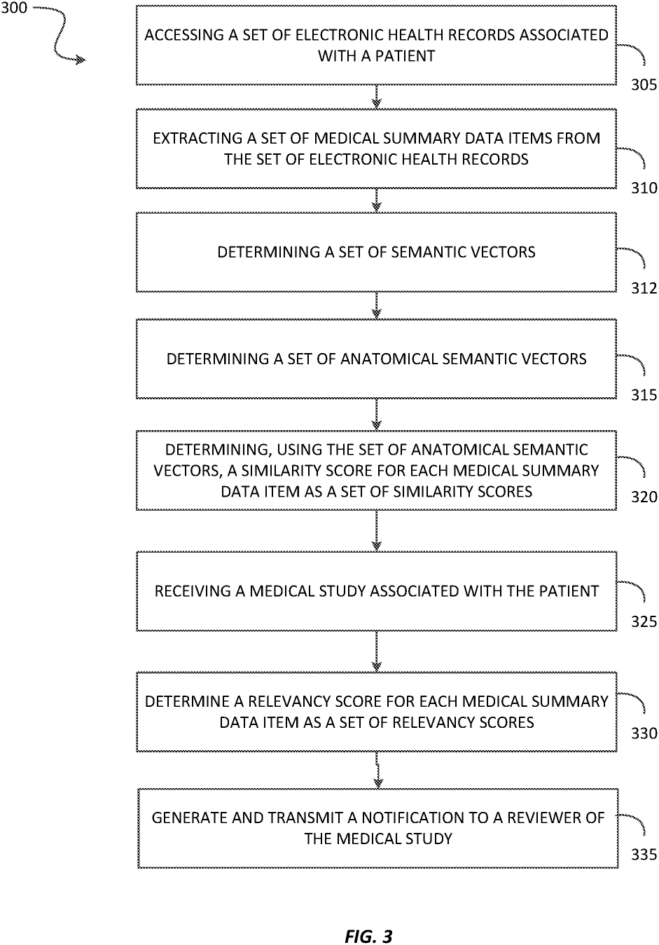

For example, FIG. 3 is a flowchart illustrating a method 300 for determining relevancy of electronic health records (including medical summary data item(s) therein) to medical analysis objectives according to some embodiments. The method 300 is described herein as being performed by the server 105 (the electronic processor 200 executing the indexing application 230). However, as noted above, the functionality performed by the server 105 (or a portion thereof) may be performed by other devices, including, for example, the user device 117 (via an electronic processor executing instructions).

As illustrated in FIG. 3, the method 300 includes receiving, with the electronic processor 200, a set of electronic health records (for example, one or more medical records 165) associated with a patient (at block 305). As noted above, in some embodiments, the medical records database 115 stores medical records 165 (for example, the set of electronic health records). In such embodiments, the electronic processor 200 receives the medical records 165 (i.e., the set of electronic health records) from the medical records database 115 over the communication network 120. Alternatively or in addition, the medical records 165 may be stored in another storage location, such as the memory of the user device 117. Accordingly, in some embodiments, the electronic processor 200 receives the medical records 165 from another storage location (for example, the memory of the user device 117). Alternatively or in addition, in some embodiments, the electronic processor 200 receives the medial record 165 (such as an imaging study) directly from the imaging modality 130 over the communication network 120. In such embodiments, the electronic processor 200 may (automatically) receive the medical record 165 upon completion of an imaging scan (including the medical record 165) of the patient by the imaging modality 130.

After receiving the set of electronic health records (for example, one or more medical records 165), the electronic processor 200 extracts a set of medical summary data items from the set of electronic health records (at block 310). As noted above, an electronic health record may include one or more medical summary data items, including, for example, symptoms, diagnoses, current or past illnesses, past surgeries, and the like. In some embodiments, the electronic processor 200 extracts the set of medical summary data items using one or more conventional approaches.

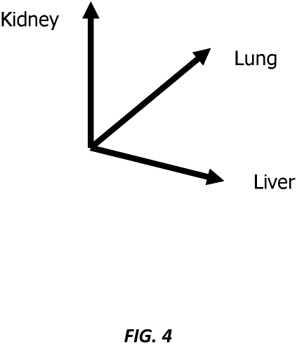

The electronic processor 200 determines a set of semantic vectors (at block 312). In some embodiments, each semantic vector represents a medical summary data item included in the set of medical summary data items. Additionally, as illustrated in FIG. 3, the electronic processor 200 then determines a set of anatomical semantic vectors (at block 315). Each anatomical semantic vector may represent at least one anatomical concept. In some embodiments, the electronic processor 200 determines the set of anatomical semantic vectors using an anatomical reference system (for example, an reference frame). In such embodiments, the electronic processor 200 may generate an anatomical reference system (or frame) including a plurality of anatomical reference vectors, where each anatomical reference vector is associated with an anatomical concept. For example, the electronic processor 200 may use a set of anatomical concepts that provide a reference frame to determine a set of anatomical semantic vectors, where each anatomical semantic vector represents at least one anatomical concept. For example, FIG. 4 illustrates an example anatomical reference system (for example, as a reference frame) according to some embodiments. As illustrated in FIG. 4, the example anatomical reference system includes a first anatomical reference vector associated with a lung (as a medical or anatomical concept), a second anatomical reference vector associated with a kidney (as a medical or anatomical concept), and a third anatomical reference vector associated with a liver (as a medical or anatomical concept).

In some embodiments, the electronic processor 200 constructs (or generates) the anatomical reference system (or frame) using selected key terms of a standard anatomical ontology, such as, for example, the Foundation Model of Anatomy. In some embodiments, the electronic processor determines a suitable (or desired) level of granularity in the ontology and anchors the reference frames at key concepts at that level (for example, lung, kidney, and liver, with reference to FIG. 4). As one example, the electronic processor 200 my use a refined anatomical ontology that includes only items or medical/anatomical concepts associated with common radiology studies (for example, generated in a process such as RadiO). Accordingly, in some embodiments, the electronic processor generates the anatomical reference system from a collection of electronic medical information (such as, for example, a large corpus of medical articles) using an established ontology.

The electronic processor 200 may then determine a similarity score for each medical summary data item as a set of similarity scores (at block 320). A similarity score represents an association between each medical summary data item and a medical or anatomical concept. In some embodiments, the electronic processor 200 determines the set of similarity scores using the set of anatomical semantic vectors. For example, in some embodiments, the electronic processor 200 using a large corpus, computes or determines semantic vectors of all medically relevant concepts (for example, those annotated to the UMLS concept system) using a standard embedding procedure. The electronic processor 200 may then map each summary item to a set of coordinates that specify the association of the concepts in the summary item with the reference concepts. The associations may be given as a function (for example, the average) of the similarity scores between the semantic vectors of the term and each reference concept. Accordingly, in some embodiments, the electronic processor 200 determines the similarity score (for example, the set of similarity scores) based on anatomical coordinates associated with the anatomical semantic vector of an associated medical summary data item. Alternatively or in addition, in some embodiments, the electronic processor 200 determines the similarity score (for example, the set of similarity scores) using a function, such as, for example, a cosine similarity function, of a semantic vector representing a corresponding medical summary data item and the anatomical semantic vector representing a corresponding anatomical concept.

In some embodiments, the electronic processor 200 stores the set of similarity scores such that each similarity score is associated with each medical summary data item (for example, as metadata). Accordingly, the set of similarity scores may be stored with each medical summary data item. Alternatively or in addition, in some embodiments, the reference concepts may be further compressed using a standard dimensionality reduction method, such as, for example, principal component analysis.

After determining the set of similarity scores, the electronic processor 200 receives a medical study associated with the patient (at block 325). The medical study may be associated with one or more medical or anatomical concepts. In some embodiments, the medical study is a new medical study, such as a medical study or imaging study recently collected by the imaging modality 130. Accordingly, in some embodiments, the electronic processor 200 receives the medical study from the imaging modality 130. Alternatively or in addition, in some embodiments, the electronic processor 200 may receive the medical study from another component of the system 100, such as for example, the user device 117, the medical record database 115, or the like.

In response to receiving the medical study associated with the patient (at block 325), the electronic processor 200 determines a relevancy score for each medical summary data item as a set of relevancy scores (at block 330). A relevancy score may represent a relevancy of each medical summary data item to the medical study. Alternatively or in addition, in some embodiments, the electronic processor 200 also considers an imaging view and may assign a set of weights on the same reference dimensions (for example, the anatomical reference system or frame). For example, each imaging view in popular radiological studies is also assigned a set of weights on the same reference dimensions. At retrieval time, summary items relevant to each view can be re-ranked on demand using the weights and the scores together. For example, using a corpus of radiology reports generated for each type of imaging study, the electronic processor 200 may extract the anatomical concepts relevant to the study. The electronic processor 200 may then map the study (and view) type to a set of weights that is a function (for example, the average) of the similarity scores between the semantic vectors of the study-specific anatomical concept and the reference concepts. The weights multiplied by the scores at each reference dimension may feed into an aggregation function (for example, summing) to produce a score for the summary item for the study-specific ranking. Additional tuning of the final score may use a more complex (for example, nonlinear, and/or trainable with user feedback) scaling function to combine the weighted scores from each dimension. Accordingly, in some embodiments, the electronic processor 200 determines the set of relevancy scores based on a set of similarity scores and a set of weights associated with an imaging view of the medical study.

As illustrated in FIG. 3, the electronic processor 200 then generates and transmits a notification to a reviewer of the medical study (at block 335). In some embodiments, the electronic processor 200 transmits the notification to the user device 117 such that the notification may be displayed via the display device 160 to a user of the user device 177, such as, for example, a reviewer of the medical study. In some embodiments, the notification indicates at least one medical summary data item that is relevant to the medical study received at block 325. Alternatively or in addition, in some embodiments, the notification includes an ordered list ranking relevant medical summary data items based on relevancy to the medical study. Accordingly, in some embodiments, the notification may function as an alert or warning to a reviewer of the medical study such that the reviewer is made aware of other medical summary data items (for example, past health data associated with the patient) that may be relevant to the current medical study being read.

Various features and advantages of the embodiments described herein are set forth in the following claims.

What is claimed is:

1. A system of determining relevancy of electronic health records to medical analysis objectives, the system comprising:
an electronic processor configured to
access a set of electronic health records associated with a patient,
extract a set of medical summary data items from the set of electronic health records,
determining a set of semantic vectors, each semantic vector representing a medical summary data item included in the set of medical summary data items,
determine, using a set of anatomical concepts providing a reference frame, a set of anatomical semantic vectors, each anatomical semantic vector representing at least one anatomical concept,
determine, using the set of anatomical semantic vectors, a similarity score for each medical summary data item as a set of similarity scores, wherein the similarity score represents an association between each medical summary data item and an anatomical concept,
receive a medical study associated with the patient, the medical study associated with at least one anatomical concept,
determine a relevancy score for each medical summary data item as a set of relevancy scores, wherein the relevancy score represents a relevancy of each medical summary data item to the medical study, and
generate and transmit a notification to a reviewer of the medical study, wherein the notification indicates at least one medical summary data item that is relevant to the medical study.

2. The system of claim 1, wherein the electronic processor is configured to generate the reference frame from a collection of electronic medical information using an established ontology.

3. The system of claim 2, wherein the established ontology is the foundational model of anatomy.

4. The system of claim 1, wherein the reference frame includes a plurality of anatomical reference vectors, wherein each anatomical reference vector is associated with an anatomical concept.

5. The system of claim 1, wherein the electronic processor is configured to determine the set of similarity scores using a function of a semantic vector representing a corresponding medical summary data item and the anatomical semantic vector representing a corresponding anatomical concept.

6. The system of claim 1, wherein the medical study is a radiology study.

7. The system of claim 1, wherein the set of medical summary data items includes structured text.

8. The system of claim 1, wherein the set of medical summary data items includes unstructured text.

9. The system of claim 1, wherein the at least one anatomical concept includes at least one selected from a group consisting of an anatomical region and an anatomical body part.

10. The system of claim 1, wherein the electronic processor is configured to determine the set of relevancy score based on a set of similarity scores and a set of weights associated with an imaging view of the medical study, wherein each weight is associated with an anatomical concept.

11. The system of claim 1, wherein the notification includes an ordered list ranking relevant medical summary data items based on relevancy to the medical study.

12. The system of claim 1, wherein the electronic health records associated with the patient are previous electronic health records associated with a medical history of the patient.

13. A method of determining relevancy of electronic health records to medical analysis objectives, the method comprising:
generating, with an electronic processor, a reference frame including a plurality of anatomical reference vectors, each anatomical reference vector associated with an anatomical concept;
accessing, with the electronic processor, a set of electronic health records associated with a patient;
extracting, with the electronic processor, a set of medical summary data items from the set of electronic health records;
determining a set of semantic vectors, each semantic vector representing a medical summary data item included in the set of medical summary data items,
determining, with the electronic processor, using the using the set of anatomical concepts providing the reference frame, a set of anatomical semantic vectors, each anatomical semantic vector representing at least one anatomical concept;
determining, with the electronic processor, using the set of anatomical semantic vectors, a similarity score for each medical summary data item as a set of similarity scores, wherein the similarity score represents an association between each medical summary data item and an anatomical concept;
receiving, with the electronic processor, a medical study associated with the patient, the medical study associated with at least one anatomical concept;
determining, with the electronic processor, a relevancy score for each medical summary data item as a set of relevancy scores, wherein the relevancy score represents a relevancy of each medical summary data item to the medical study; and
generating and transmitting, with the electronic processor, a notification to a reviewer of the medical study, wherein the notification indicates at least one medical summary data item that is relevant to the medical study.

14. The method of claim 13, wherein determining the similarity score includes determining the similarity score using a function of a semantic vector representing a corresponding medical summary data item and the anatomical semantic vector representing a corresponding anatomical concept.

15. The method of claim 13, wherein receiving the medical study includes receiving a radiology study.

16. The method of claim 13, wherein determining the set of relevancy scores includes determining the set of relevancy scores based on the set of similarity scores and a set of weights associated with an imaging view of the medical study, wherein each weight is associated with an anatomical concept.

17. The method of claim 13, wherein generating and transmitting the notification includes generating and transmitting a notification including an ordered list ranking relevant medical summary data items based on relevancy to the medical study.

18. A non-transitory, computer-readable medium storing instructions that, when executed by an electronic processor, perform a set of functions, the set of functions comprising:
accessing a set of electronic health records associated with a patient;
extracting a set of medical summary data items from the set of electronic health records;
determining a set of semantic vectors, each semantic vector representing a medical summary data item included in the set of medical summary data items,
determining, using a set of anatomical concepts providing a reference frame, a set of anatomical semantic vectors, each anatomical semantic vector representing at least one anatomical concept;
determining, using the set of anatomical semantic vectors, a similarity score for each medical summary data item as a set of similarity scores, wherein the similarity score represents an association between each medical summary data item and an anatomical concept;
receiving a radiology study associated with the patient, the medical study associated with at least one anatomical concept;
determining a relevancy score for each medical summary data item as a set of relevancy scores, wherein the relevancy score represents a relevancy of each medical summary data item to the radiology study; and
generating and transmitting a notification to a reviewer of the medical study, wherein the notification indicates at least one medical summary data item that is relevant to the radiology study.

19. The computer readable medium of claim 18, wherein determining the similarity score includes determining the similarity score using a function of a semantic vector representing a corresponding medical summary data item and the anatomical semantic vector representing a corresponding anatomical concept.

20. The computer readable medium of claim 18, wherein determining the set of relevancy scores includes determining the set of relevancy scores based on the set of similarity scores and a set of weights associated with an imaging view of the medical study, wherein each weight is associated with an anatomical concept.
